(19) United States
(12) Patent Application Publication
(10) Pub. No.: US 2022/0130544 A1
(43) Pub. Date: Apr. 28, 2022

(54) MACHINE LEARNING TECHNIQUES TO ASSIST DIAGNOSIS OF EAR DISEASES

(71) Applicant: Remmie, Inc

(72) Inventors: Jane Yuqian Zhang, Bothell, WA (US); Zhan Wang, Newcastle, WA (US)

(21) Appl. No.: 17/508,517

(22) Filed: Oct. 22, 2021

Related U.S. Application Data
(60) Provisional application No. 63/104,932, filed on Oct. 23, 2020.

(51) Int. Cl. G16H 50/20; G16H 30/20

(52) U.S. Cl. CPC G16H 50/20; G16H 30/20

(57) ABSTRACT

Various aspects of methods, systems, and use cases may be used to generate an ear disease state prediction to assist diagnosis of an ear disease. A method may include receiving an image an ear, predicting an image-based confidence level of a disease state in the ear by using the image as in input to a machine learning trained model. The method may include receiving text, for example corresponding to a symptom of the patient. The method may include predicting a symptom-based confidence level of the disease state in the ear by using the text as in input to a trained classifier. The method may include using the results of the image-based confidence level and the symptom-based confidence level to determine an overall confidence level of presence of an ear infection in the ear of the patient.

### CLAIM OF PRIORITY

This application claims the benefit of priority to U.S. Provisional Application No. 63/104,932 filed Oct. 23, 2020, titled “SYSTEM: AND METHOD OF USING MACHINE LEARNING BASED ALGORITHM TO ASSIST REMOTE DIAGNOSIS OF EAR, NOSE, THROAT AND UPPER RESPIRATORY TRACT DISEASES,” which is hereby incorporated herein by reference in its entirety.

### BACKGROUND

Acute Otitis Media (AOM, or ear infection) is the most common reason for a sick child visit in the US as well as low to mid income countries. Ear infections account for the most common reason for antibiotics usage for children under 6 years, particularly in the 24-month to 3 age group. It is also the second most important cause of hearing loss, impacting 1.4 billion in 2017 and ranked fifth highest disease burden globally.

During a physician's visit, the standard practice for diagnosing an AOM requires inserting an otoscope with a disposable speculum in the external ear along the ear canal to visualize the tympanic membrane (eardrum). A healthy eardrum appears clear and pinkish-gray, whereas an infected one will appear red and swollen due to fluid buildup behind the membrane. Access to otolaryngology, pediatric, or primary specialist is severely limited in low resource settings, leaving AOM undiagnosed or misdiagnosed. The primary unmet needs with an ear infection are the lack of means to track disease progression, which could lead to delayed diagnosis at onset or ineffective treatment.

During a physician's visit, an otoscope with a disposable speculum is inserted in the external ear along the ear canal to visualize the tympanic membrane (eardrum). A healthy eardrum appears clear and pinkish-gray, whereas an infected one will appear red and swollen due to fluid buildup behind the membrane. However, these features are not immediately distinguishable especially when there is limited time to view the eardrum especially of a squirmy child using a traditional otoscope,

Telemedicine provides a viable means for in-home visits to a provider with no wait time and closed-loop treatment guidance or prescription, An ear infection is an ideal candidate for real-time telemedicine visits, but due to the lack of means to visualize inside the ear, telemedicine provider cannot accurately diagnose an ear infection. As a result, telemedicine was found to lead to over-prescription of antibiotics or “new utilization” of clinical resources which would otherwise not occur compared to in-person visits.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a platform for ear nose and throat disease state diagnostic support in accordance with at least one example of this disclosure,

FIG. 2 illustrates a system for training and implementing a model and classifier for predicting outcomes related to ear nose and throat disease state in accordance with at least one example of this disclosure.

FIG. 3A illustrates examples of a healthy eardrum and an infected eardrum in accordance with at least one example of this disclosure.

FIG. 3B illustrates an example of data augmentation to generate training data in accordance with at least one example of this disclosure.

FIG. 3C illustrates an example of image segmentation in accordance with at least one example of this disclosure.

FIGS. 4A-4B illustrate results of image and text classification predictions in accordance with at least one example of this disclosure.

FIG. 5 illustrates a flowchart showing a technique for generating an ear disease state prediction to assist diagnosis of an ear disease in accordance with at least one example of this disclosure.

FIG. 6 illustrates a block diagram of an example machine upon which any one or more of the techniques discussed herein may perform in accordance with at least one example of this disclosure.

### DETAILED DESCRIPTION

A system and method for early and remote diagnosis of ear disease is disclosed. An images of a patient's inner ear may be taken with an otoscope and transmitted to a cloud-based database. A machine learning-based algorithm is used to classify images for presence or absence of diseases such as AOM, and other diagnosis. The results of the classification and diagnosis may be sent to third parties such as physician, healthcare providers to be integrated in patient care decisions.

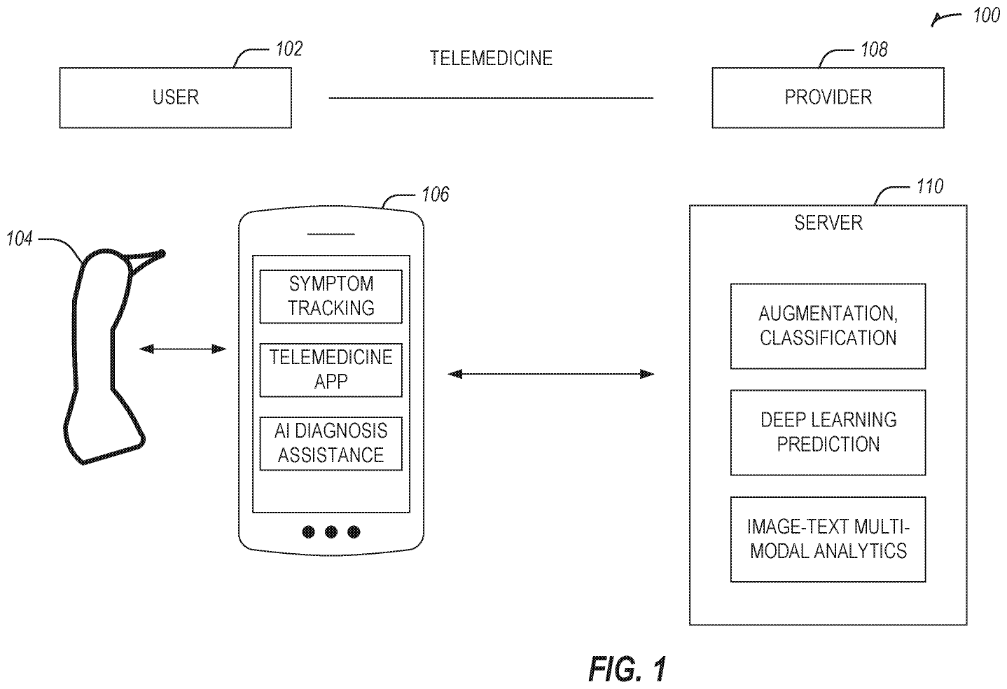

An otitis media is most commonly diagnosed using an otoscope (FIG. 1), essentially a light source with a magnifying eyepiece for visualization of the ear canal and eardrum with the human eye. The key features of these currently commercially available products are summarized in Tablet These otoscopes lack communication functions requisite for the current invention but can be incorporated after the communication functions are fulfilled by complementing devices.

In one embodiment, an otoscope is disclosed that is configured to be used together with a host device, such as a smart phone or other handheld mobile devices. The host device can be used to capture images. The images can be uploaded to cloud-based database. The images can be shared through an app in the host device. The uploaded images are labelled with respective clinical diagnosis.

In one embodiment, the uploaded images can be used as data source to train the algorithm. At least 500 “normal”, 300 AOM images, and additional images with “other” ailments (O/W, OME, and CSOM) are collected for training purposes. The images are de-identified and securely stored for subsequent analysis.

In normal operation of an otoscope, the eardrum may be visualized in varying regions of the field of view. Translation of images will make the algorithm location invariant.

FIG. 1 illustrates a platform 100 for ear nose and throat disease state diagnostic support in accordance with at least one example of this disclosure. The platform 100 includes a user ecosystem 102 and a provider ecosystem 108. The two ecosystems 102 and 108 may perform various functions, with some overlap and some unique to the ecosystem. In some examples, the user ecosystem 102 and the provider ecosystem 108 are remote from each other (e.g., a patient may be at home using the user ecosystem 102, while a doctor operates the provider ecosystem 108 from an office), and in other examples the ecosystems 102 and 108 may be local to each other, such as when a patient visits a doctor's office. The devices of the user ecosystem 102 and the provider ecosystem 108 may communicate (e.g., via a network, wirelessly, etc.) with each other and with devices within each ecosystem.

In an example, the user ecosystem 102 includes an otoscope 104 and a user device 106 (e.g., a mobile device such as a phone or a tablet, a computer such as a laptop or a desktop, a wearable, or the like). The otoscope 104 may be communicatively coupled to the user device 106 (e.g., configured to send data such as an image over a wired or wireless connection, such as Bluetooth, Wi-Fi, Wi-Fi direct, near field communication (NFC), or the like). In some examples, functionality of the otoscope 104 may be controlled by the user device 106. For example, the user device 106 may trigger a capture of an image or video at the otoscope 104. The triggering may be caused by a user selection on a user interface on the user device 106, caused automatically (e.g., via a detection of an object within a camera view of the otoscope 104, such as an ear drum), or via remote action (e.g., by a device of the provider ecosystem 108). When the trigger is via a remote action, the remote action may include a provider selection on a user interface of a device of the provider ecosystem 108 indicating that the camera view of the otoscope 104 is acceptable (e.g., a capture will include an image of an ear drum or other anatomical feature of a patient).

The otoscope 104 may be used to capture an image of an ear drum or inner ear portion of a patient. When the image is captured, it may be sent to the user device 106, which may in turn send the image a device of the provider ecosystem 108, such as a server 110. In another example, the image may be sent directly from the otoscope 104 to the server 110. The user device 106 may receive an input including text on a user interface by a patient in some examples, such as user entered text, a selection of a menu item, or the like. The user input may include a text representation of a symptom (e.g., fever, nausea, sore throat, etc.). The user input may be sent from the user device 106 to the server 110.

The user device 106 may be used to track symptoms, place or receive secure calls or send or receive secure messages to a provider, or perform AI diagnostic assistance. The server 110 may be used to place or receive secure calls or send or receive secure messages with the user device 106. The server 110 may perform augmentation classification to train a model (e.g., the AI diagnosis assistant), use a model to perform a deep learning prediction, or perform image-text multi-modal analytics. In some examples, the server 110 or the user device 106 may output a prediction for diagnosis assistance, such as a likelihood of a patient having an ear infection. The prediction may be based on images captured by the otoscope 104 input to a deep learning model. The prediction may be based on text received via user input at the user device 106 (or over a phone call with a provider, entered by the provider) input to a text classifier. The prediction may be based on an output of both the deep learning model and the text classifier (e.g., a combination, such as by multiplying likelihoods together, taking an average likelihood, using one of the results as a threshold, etc.).

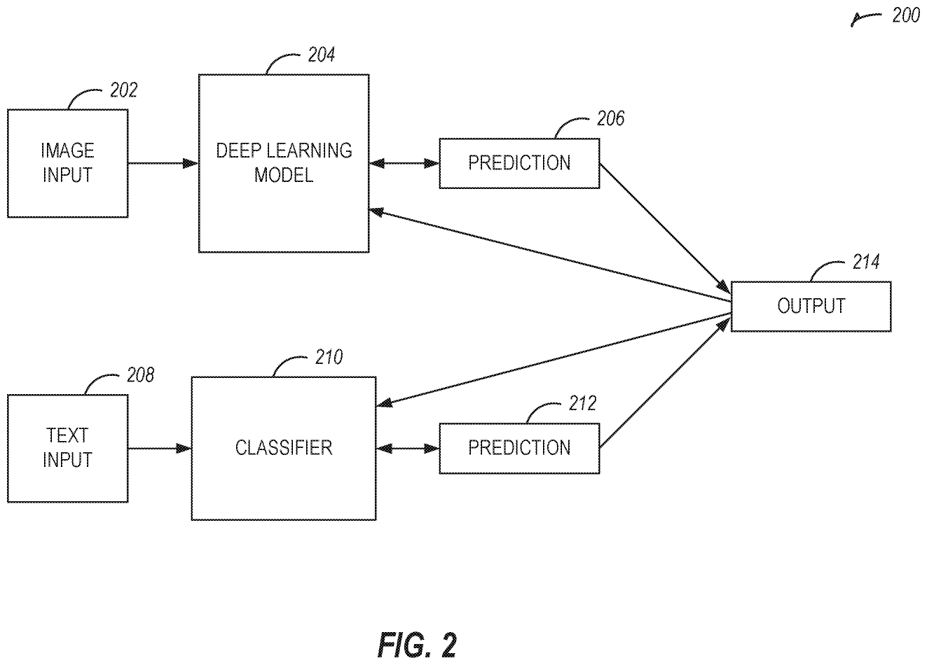

FIG. 2 illustrates a block diagram 200 for training and implementing a model and classifier for predicting outcomes related to ear nose and throat disease state in accordance with at least one example of this disclosure. The block diagram 200 includes a deep learning model 204 and a classifier 210, which each receive inputs and output a prediction. The deep learning model 204 receives an image input 202 and outputs an image-based prediction 206 and the classifier 210 receives a text input 208 and outputs a text-based prediction 212. The image-based prediction 206 and the text-based prediction 212 may be combined as an output 214. The output may include either prediction 206 or 212, or a combination, such as by multiplying likelihoods together, taking an average likelihood, using one of the results as a threshold, or the like.

In some examples, the prediction 206 may be fed back to the deep learning model 204, for example as a label for the image input 202 when training the deep learning model 204. In another example, the prediction 206 may be fed back to the deep learning model 204 as a side input (e.g., for use in a recurrent neural network). The output 214 may be used similarly to the prediction 206 for feedback or training purposes. The prediction 212 may be used similarly with the classifier 210.

In an example, images may be represented as a 2D grid of pixels. CNNs as the deep learning network may be used for data with a grid-like structure.

Applying CNNs in the case of diagnosis, a medical image may be analyzed for a binary classification or a probability problem, giving the ill versus normal and the likelihood of illness as a reference for a doctor's decision,

Image only techniques may be improved with new architecture with additional layers, more granular features on the images, and optimized weights in custom model specific for AOM that may be deployed over mobile computing.

In one embodiment, Tensorflow (of Google) architecture may be used for the deep learning-based image classification (e.g., for implementing the deep learning model 204). A set of proprietary architecture components including selected model type, loss function, batch size, and a threshold may be used as input for classification predictions. The selection criteria of the architecture components may include optimal performance in recall and precision and real number metric values were easy to translate and manipulate for mixing with text classification.

Testing on the validation dataset may include an F1 value of 72% for image classification in which F1 value is defined as a tradeoff between precision and recall.

A multi-model model using a TensorFlow model may be used to achieve more accurate results. In one embodiment, the multi-model classification (e.g., at the classifier 210) combines image and text classification, mixing their confidence values and generate a new decision based on threshold.

In the above mentioned multi-model classification embodiment (e.g., classifier 210), a grid search method may be used with two parameters for improved performance including a weight of image and text results (e.g., how much is the image used and text, respectively), or setting of a threshold for making a binary classification. For example, when the combined confidence value, such as probability is 0.7, setting threshold to 0.6 and 0.8 may yield opposite decisions.

In an example, one challenge includes using short text classification with very limited context. In an example, users may choose from a given set of symptoms. Although they are short texts, the vocabulary may be confined to a small set, for example considering some symptoms that are specifically for a particular illness. That is, if for all or most of the ill cases, some symptoms exist in the training dataset, and exclusively not in the normal case data. The classifier to make these symptoms may include strong indicators of the illness for drawing conclusions with high confidence values.

In an example, a support vector machine (SVM) may be used as a text classification algorithm for the classifier 210. In some examples, the SVM may have a difficult output to interpret or combine with the result from the image model. A logistic regression may be chosen as a tool for text classification because it is easy to implement and interpret. This may help better design the symptom descriptions.

The AI diagnosis assistant system uses the deep learning model 204, for example with a convolutional neural network (CNN).

In an example, an image classification may be performed on the input image 202 using the deep learning model. An object detection technique may be used on the input image, for example before it is input to the deep learning model. The object detection may be used to determine whether the image properly captured an eardrum or a particular area of an eardrum. For example, the object detection may detect a Malleus Handle on a captured eardrum. After detecting the Malleus Handle, the image may be segmented (e.g., with two perpendicular lines to create four quadrants). The segmented portions of the image may be separately classified with the deep learning model as input images in some examples.

Another object detection may be used, together (before, after, or concurrently) or separately from the above object detection. This object detection may include detecting whether a round or ellipse shaped eardrum appears in a captured image. This object detection may include determining whether the round or ellipse shaped eardrum occupies at least a threshold percentage (e.g., 50%, 75%, 90%, etc.) of the captured image. In some examples, clarity or focus of the image (e.g., of the round or ellipse shaped eardrum portion of the image) may be checked during object detection.

In some examples, a set of deep learning trained models may be used. For example, a different model may be used for each segmented portion of an image. In an example where a captured image is segmented into four quadrants based on object detection of a Malleus Handle, four models may be trained or used. An output of a deep learning model may include a number, such as a real number between 0 and 1. When using more than one model, a prediction indication may be generated based on values from a set of or all of the models used. example, an average, medium, or other combination of model output numbers may be used to form a prediction. The prediction may indicate a percentage likelihood of a disease state of a patient (e.g., an ear infection in an ear or a portion of an ear).

Data may be collected for training the deep learning model 204 from consenting patients, in some examples. An image may be captured of a patient, such as by a clinician (e.g., a doctor), by the patient, by a caretaker of the patient, or the like. The image may be labeled with an outcome, such as a diagnosis from a doctor (e.g., that an ear infection was present). In some examples, other data may be collected from the patient, such as symptoms. The other data may be used as an input to the classifier 210. An output of the classifier (e.g., prediction 212) may be used to augment the output of the deep learning model, as discussed further below. The other data may be selected by a patient, caretaker, or clinician, such as by text input, text drop down selection on a user interface, spoken audio to text capture, or the like. The image and text data may be captured together (e.g., during a same session using an application user interface) or separately (e.g., text at an intake phase, and an image at a diagnostic phase).

A system may be trained using a multi-modal approach, including image and text classification. For an image model (e.g., deep learning model 204), one or more CNNs may be used, for example. For the classifier 210, in an example, a support vector machine classifier, naive bayes algorithm, or other text classifier may be used. The deep learning model 204 and the classifier 210 may output separate results (e.g., predictions 206 and 212 of likelihood of the presence of a disease state, such as an ear infection). The separate results may be combined, such as by multiplying percentage predictions, using an average, using one as a confirmation or threshold for the other (e.g., not using the text output if the image input is below a threshold), or the like as the output 214.

During an inference use of the deep learning model 204 and the classifier 210, a user may receive a real time or near real time prediction of a disease state for use in diagnosis. The inference may be provided to a user locally or remotely. In the local example, a doctor may capture an image of a patient, and text may be input by the patient or the doctor. The doctor may then view the prediction, which may be used to diagnose the patient. In the remote example, the patient may capture the image and input the text, which may be used to generate the inference. In the remote example, the inference may be performed at a patient device, at a doctor operated device, or remote to both the doctor and the patient (e.g., at a server). The results may be output for display on the doctor operated device (e.g., a phone, a tablet, a dedicated diagnosis device, or the like). The doctor may then communicate a diagnosis to the patient, such as via input in an application which may be sent to the patient, via a text message, via a phone call, via email, etc. In the remote example, a doctor may view a patient camera (e.g., an otoscope) live. In an example, the doctor may cause capture of an image at the doctor's discretion. In another example, the patient may record video, which the doctor may use to capture an image at a later time.

In a real-time consult example, a user may stream video to a doctor and the doctor may take a snapshot image. The doctor may receive an input symptom description from the patient. A UI component (for example, a button) may be used to allow the doctor to query the model to perform a prediction for the possibility of an ear infection or other disease state. In another example, the user may capture an image or input symptoms before the doctor consultant and send the information to the doctor. The doctor may import the data to the model or ask for the prediction.

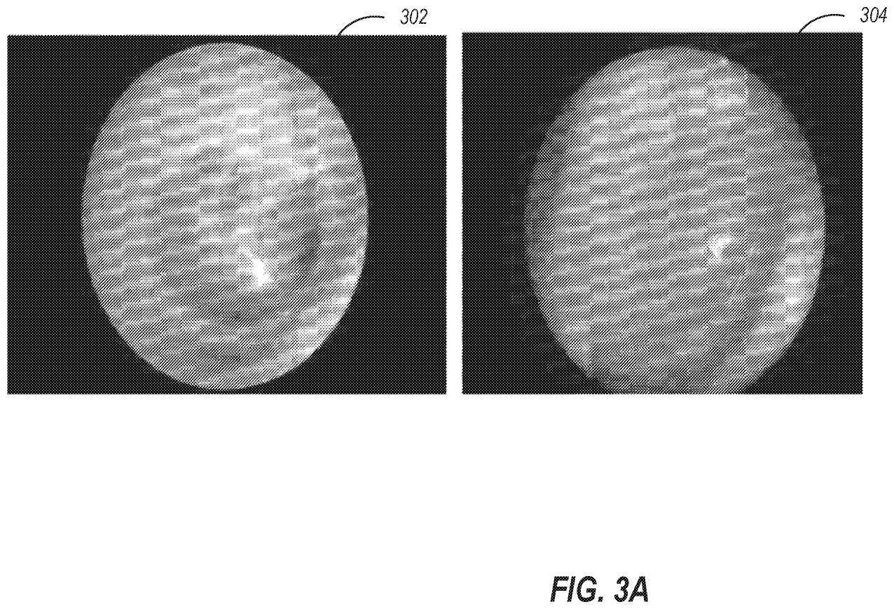

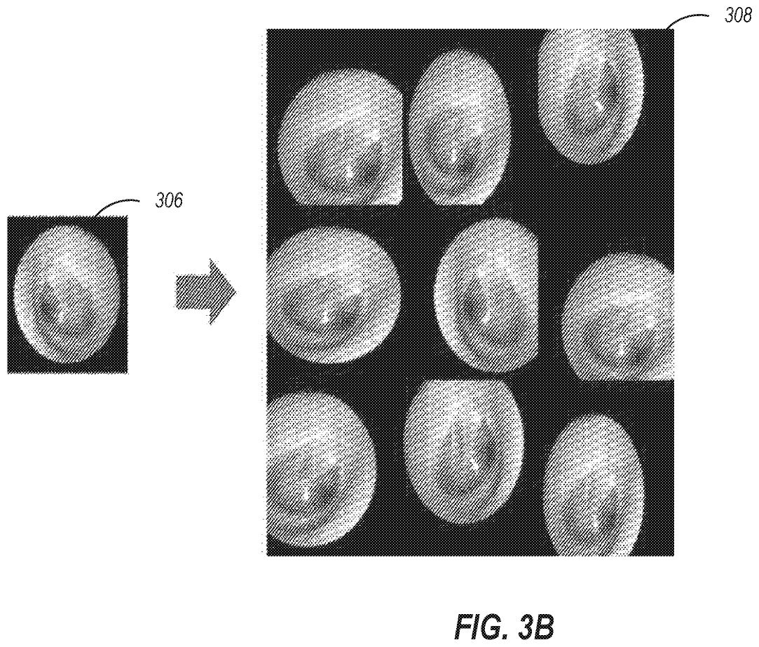

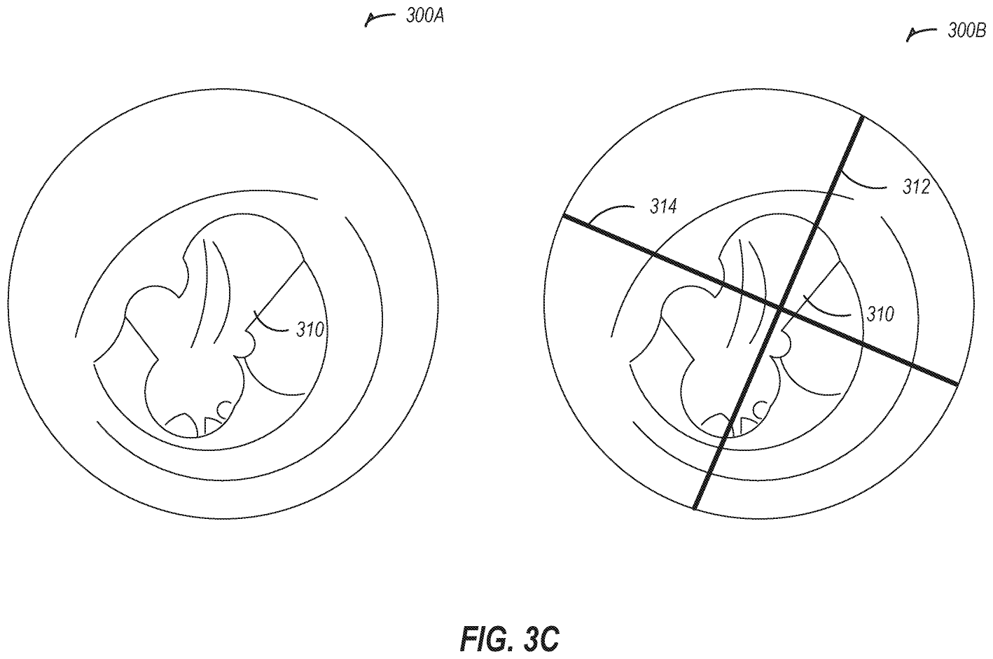

FIG. 3A illustrates examples of a healthy eardrum and an infected eardrum in accordance with at least one example of this disclosure.

A healthy eardrum appears clear and pinkish-gray, whereas an infected one will appear red and swollen due to fluid buildup behind the membrane.

FIG. 3B illustrates an example of data augmentation to generate training data in accordance with at least one example of this disclosure.

Data augmentation may be used to create a larger dataset for training the algorithm. In one embodiment, a combination of several data augmentation approaches is adopted, including translation, rotation and scaling. Additional augmentation methods, said color and brightness adjustments, are introduced if needed.

In one embodiment, by using python library of Keras (https://keras.io/) an original image can generate 10 images through rotating, flipping, contrast stretching, histogram equalization, etc. The new images still retain the underlying patterns among pixels and serve as random noises to help train the classifier.

An eardrum may be visualized in several orientations and by augmenting the training data with rotated examples the algorithm will be robust to changes in rotation.

The actual size of eardrum changes as a patient grows and varies from patient to patient, additionally, the size of the eardrum in an otoscope image will vary depending on the position of the device in the ear. The image dataset may be augmented by using scaling to make the algorithm robust to images of varying size.

FIG. 3C illustrates an example of image segmentation in accordance with at least one example of this disclosure. The image segmentation may include an object detection, which is shown in a first image 300A of an ear of a patient. The object detection may be used to identify a Malleus Handle or other anatomical feature at location 310 of an ear drum of the ear of the patient. After identification of the Malleus Handle or other anatomical feature, the image may be segmented, for example into quadrants. The quadrants may be separated according to a line 312 (which may not actually be drawn, but is shown for illustrative purposes in a second image 300B of FIG. 3C) that bisects, is parallel to, or otherwise references the Malleus Handle or other anatomical feature. A second line 314 (again, shown in the second image 300B of FIG. 3C, but not necessarily drawn on the image in practice) may be used to further segment the image into the quadrants by bisecting the line 312, for example, or otherwise intersecting with the line 312. Further segmentation may be used (e.g., additional lines offset from the lines 312 or 314) in some examples. Each portion of the segmented image in 300B may be used with a model (e.g., a separate model or a unified model) for detecting disease state as described herein.

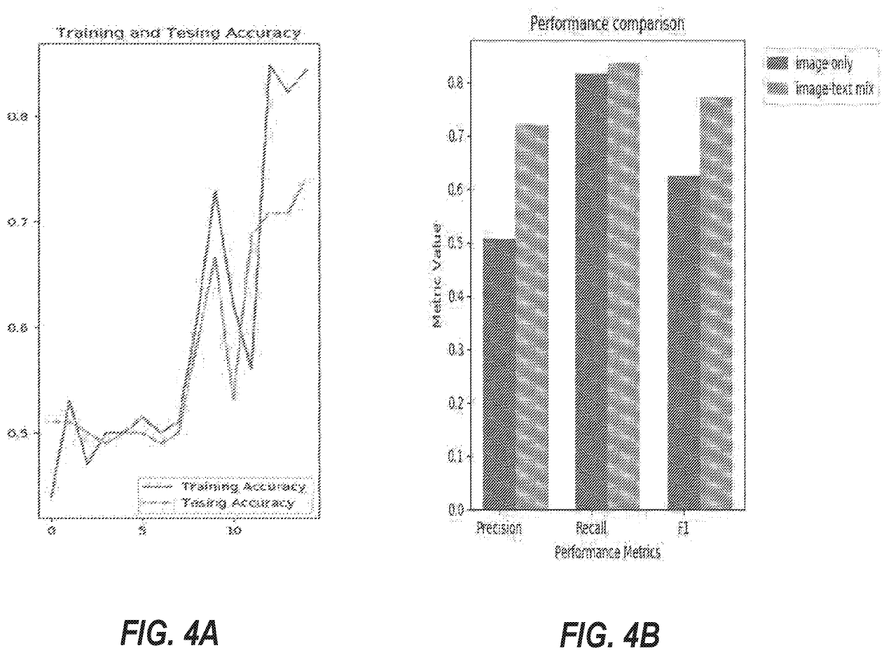

FIGS. 4A-4B illustrate results of image and text classification predictions in accordance with at least one example of this disclosure.

The classification of eardrum images is complicated. Off-the-shelf models, such as AWS Rekognition (of Amazon) and Azure CustomVision (of Microsoft) may be used for testing. Both services yield high accuracy.

As FIG. 4A shows, the model (e.g., combining image and text classification) training tends to converge well with a low training loss and evaluation accuracy reaches above 70%.

Testing on the validation dataset yields an F1 value of 72% for image classification in which F1 value is defined as a tradeoff between precision and recall. As shown in FIG. 4B, the multi-model classification brings up the overall accuracy from original 72%) to over 90%. This proves the effectiveness of the multi-model classification method.

Once the device collects sufficient data, the data may be used to train selected off-the-shelf models and further develop the custom model.

in order to select one off-the-shelf model that provides the best Positive Predicated Value (Precision) and Sensitivity (Recall), 500 normal and 300-500 AOM images are tested in off-the-shelf models to compare and contrast performance. In some embodiments, off-the-shelf models adopted include Alexnet, GoogLeNet, ResNet, Inception-V3, SqueezeNet, MobileNet-V2, public packages Microsoft Custom Vision, or Amazon Rekognition.

Transfer learning may be used to build the custom architecture. In one embodiment, 500 validation images with blinded labels are used to test the algorithm for at least 90% PPV and 95% sensitivity in identifying an AOM. An iterative approach may be taken once additional training images become available to optimize the algorithm.

In one embodiment, the algorithm may be built-in to an app used for clinical validation to classify, for example, at least 50 new test images, blinded against clinical diagnosis by a provider. A usability interview may be conducted to collect feedback from the provider regarding User Experience Design and result interpretation of the model output for future improvement.

In some embodiments, the algorithm may be used to support diagnosis of other ear, nose, and throat ailments for adults and children. In performing expansion of the classification to identify images not classified as normal or AOM, including but not limited to Obstructing Wax or Foreign Bodies (O/W), Otitis Media with Effusion (OME), or Chronic Suppurative Otitis Media with Perforation (CSOM with Perforation). Image augmentation may increase the training data size. A similar iterative process may be performed, characterized, compared, or optimized as that for AOM.

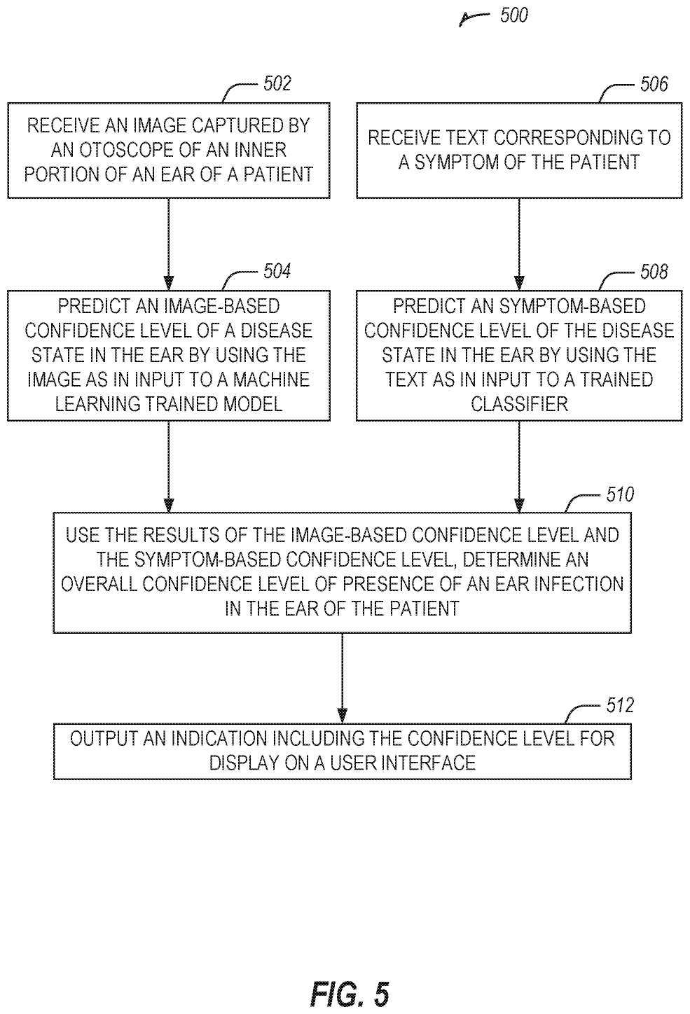

FIG. 5 illustrates a flowchart showing a technique 500 for generating an ear disease state prediction to assist diagnosis of an ear disease in accordance with at least one example of this disclosure. The technique 500 may be performed by a processor by executing instructions stored in memory.

The technique 500 includes an operation 502 to receive an image captured by an otoscope of an inner portion of an ear of a patient. The technique 500 includes an operation 504 to predict an image-based confidence level of a disease state in the ear by using the image as in input to a machine learning trained model. The machine learning trained model may include a convolutional neural network model.

The technique 500 includes an operation 506 to receive text corresponding to a symptom of the patient. In an example, receiving the text may include receiving a selection from a list of symptoms, In another example, receiving the text may include receiving user input custom text.

The technique 500 includes an operation 508 to predict a symptom-based confidence level of the disease state in the ear by using the text as in input to a trained classifier. The trained classifier may include a support vector machine (SVM) classifier or a logistic regression model classifier.

The technique 500 includes an operation 510 to use the results of the image-based confidence level and the symptom-based confidence level to determine an overall confidence level of presence of an ear infection in the ear of the patient. In an example, the overall confidence level may include a confidence level output from the machine learning trained model multiplied by a confidence level output from the trained classifier. In other examples, the overall confidence level may include an average of the confidence level output from the machine learning trained model and the confidence level output from the trained classifier. In some examples, the overall confidence level may use one of the confidence level output from the machine learning trained model and the confidence level output from the trained classifier as a threshold, and output the other. The technique 500 includes an operation 512 to output an indication including the confidence level for display on a user interface,

The technique 500 may include segmenting the image, and wherein the input to the machine learning trained model includes each segmented portion of the image. In this example, the technique 500 may include performing object detection on the image to identify a Malleus Handle in the image, and wherein segmenting the image includes using the identified Malleus Handle as an axis for segmentation. In an example, the technique 500 may include performing object detection on the image to identify whether the image captures an entirety of an ear drum of the ear.

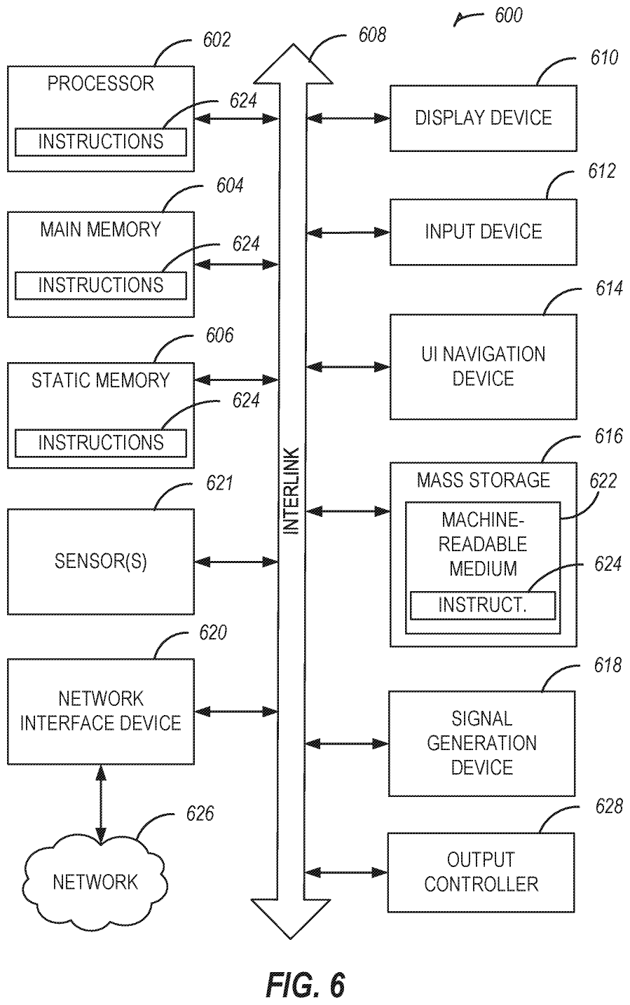

FIG. 6 illustrates a block diagram of an example machine 600 upon which any one or more of the techniques discussed herein may perform in accordance with some embodiments. In alternative embodiments, the machine 600 may operate as a standalone device and/or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine (e.g., computer system) 600 may include a hardware processor 602 (e.g., a central processing unit (CPU), a graphics processing unit (GPU), a hardware processor core, or any combination thereof), a main memory 604 and a static memory 606, some or all of which may communicate with each other via an interlink (e.g., bus) 608. The machine 600 may further include a display unit 610, an alphanumeric input device 612 (e.g., a keyboard), and a user interface (UI) navigation device 614 (e.g., a mouse), In an example, the display unit 610, input device 612 and UI navigation device 614 may be a touch screen display. The machine 600 may additionally include a storage device (e.g., drive unit) 616, a signal generation device 618 (e.g., a speaker), a network interface device 620, and one or more sensors 621, such as a global positioning system (GPS) sensor, compass, accelerometer, or other sensor. The machine 600 may include an output controller 628, such as a serial (e.g., Universal Serial Bus (USB), parallel, or other wired or wireless (e.g., infrared (IR), near field communication (NFC), etc.) connection to communicate and/or control one or more peripheral devices (e.g., a printer, card reader, etc.).

The storage device 616 may include a machine readable medium 622 on which is stored one or more sets of data structures or instructions 624 (e.g., software) embodying or utilized by any one or more of the techniques or functions described herein. The instructions 624 may also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 616 may constitute machine readable media.

While the machine readable medium 622 is illustrated as a single medium, the term “machine readable medium” may include a single medium or multiple media (e.g., a centralized or distributed database, and/or associated caches and servers) configured to store the one or more instructions 624. The term “machine readable medium” may include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine-readable medium examples may include solid-state memories, and optical and magnetic media.

The instructions 624 may further be transmitted or received over a communications network 626 using a transmission medium via the network interface device 620 utilizing any one of a number of transfer protocols (e.g., frame relay, interne protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 600, and includes digital or analog communications signals or other intangible medium to facilitate communication of such software.

Each of the following non-limiting examples may stand on its own, or may be combined in various permutations or combinations with one or more of the other examples.

Example 1 is a method for generating an ear disease state prediction to assist diagnosis of an ear disease, the method comprising: receiving, at a processor, an image captured by an otoscope of an inner portion of an ear of a patient; predicting, at the processor, an image-based confidence level of a disease state in the ear by using the image as in input to a machine learning trained model; receiving text corresponding to a symptom of the patient; predicting a symptom-based confidence level of the disease state in the ear by using the text as in input to a trained classifier; using the results of the image-based confidence level and the symptom-based confidence level to determine an overall confidence level of presence of an ear infection in the ear of the patient; and outputting an indication including the confidence level for display on a user interface.

In Example 2, the subject matter of Example 1 includes, segmenting the image, and wherein the input to the machine learning trained model includes each segmented portion of the image.

In Example 3, the subject matter of Example 2 includes, performing object detection on the image to identify a Malleus Handle in the image, and wherein segmenting the image includes using the identified Malleus Handle as an axis for segmentation.

In Example 4, the subject matter of Examples 1-3 includes, performing object detection on the image to identify whether the image captures an entirety of an ear drum of the ear.

In Example 5, the subject matter of Examples 1-4 includes, wherein determining the overall confidence level includes multiplying a confidence level output from the machine learning trained model by a confidence level output from the trained classifier.

In Example 6, the subject matter of Examples 1-5 includes, wherein receiving the text includes receiving a selection from a list of symptoms.

In Example 7, the subject matter of Examples 1-6 includes, wherein receiving the text includes receiving user input custom text.

In Example 8, the subject matter of Examples 1-7 includes, wherein the classifier is a support vector machine (SVM) classifier or a logistic regression model classifier.

In Example 9, the subject matter of Examples 1-8 includes, wherein the machine learning trained model is a convolutional neural network model.

Example 10 is a system for generating an ear disease state prediction to assist diagnosis of an ear disease, the system comprising: processing circuitry; and memory including instructions, which when executed, cause the processing circuitry to: receive, at a processor, an image captured by an otoscope of an inner portion of an ear of a patient; predict, at the processor, an image-based confidence level of a disease state in the ear by using the image as in input to a machine learning trained model; receive text corresponding to a symptom of the patient; predict a symptom-based confidence level of the disease state in the ear by using the text as in input to a trained classifier; use the results of the image-based confidence level and the symptom-based confidence level to determine an overall confidence level of presence of an ear infection in the ear of the patient; and output an indication including the confidence level for display on a user interface.

In Example 11, the subject matter of Example 10 includes, wherein the instructions further cause the processing circuitry to segment the image, and wherein the input to the machine learning trained model includes each segmented portion of the image.

In Example 12, the subject matter of Example 11 includes, wherein the instructions further cause the processing circuitry to perform object detection on the image to identify a Malleus Handle in the image, and wherein segmenting the image includes using the identified Malleus Handle as an axis for segmentation.

In Example 13, the subject matter of Examples 10-12 includes, wherein the instructions further cause the processing circuitry to perform in object detection on the image to identify whether the image captures an entirety of an ear drum of the ear.

In Example 14, the subject matter of Examples 10-13 includes, wherein to determine the overall confidence level, the instructions further cause the processing circuitry to multiply a confidence level output from the machine learning trained model by a confidence level output from the trained classifier.

In Example 15, the subject matter of Examples 10-14 includes, wherein to receive the text, the instructions further cause the processing circuitry to receive a selection from a list of symptoms.

In Example 16, the subject matter of Examples 10-15 includes, wherein to receive the text, the instructions further cause the processing circuitry to receive user input custom text.

In Example 17, the subject matter of Examples 10-16 includes, wherein the classifier is a support vector machine (SVM) classifier or a logistic regression model classifier.

In Example 18, the subject matter of Examples 10-17 includes, wherein the machine learning trained model is a convolutional neural network model.

Example 19 is at least one machine-readable medium including instructions for generating an ear disease state prediction to assist diagnosis of an ear disease, which when executed by processing circuitry, cause the processing circuitry to perform operations to: receive, at a processor, an image captured by an otoscope of an inner portion of an ear of a patient; predict, at the processor, an image-based confidence level of a disease state in the ear by using the image as in input to a machine learning trained model; receive text corresponding to a symptom of the patient; predict a symptom-based confidence level of the disease state in the ear by using the text as in input to a trained classifier; determine, using the results of the image-based confidence level and the symptom-based confidence level, an overall confidence level of presence of an ear infection in the ear of the patient; and output an indication including the confidence level for display on a user interface.

In Example 20, the subject matter of Example 19 includes, wherein the classifier is a support vector machine (SVM) classifier or a logistic regression model classifier and wherein the machine learning trained model is a convolutional neural network model.

Example 21 is at least one machine-readable medium including instructions that, when executed by processing circuitry, cause the processing circuitry to perform operations to implement of any of Examples 1-20.

Example 22 is an apparatus comprising means to implement of any of Examples 1-20.

Example 23 is a system to implement of any of Examples 1-20.

Example 24 is a method to implement of any of Examples 1-20.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

What is claimed is:

1. A method for generating an ear disease state prediction to assist diagnosis of an ear disease, the method comprising:
receiving, at a processor, an image captured by an otoscope of an inner portion of an ear of a patient;
predicting, at the processor, an image-based confidence level of a disease state in the ear by using the image as in input to a machine learning trained model;
receiving text corresponding to a symptom of the patient;
predicting a symptom-based confidence level of the disease state in the ear by using the text as in input to a trained classifier;
using the results of the image-based confidence level and the symptom-based confidence level to determine an overall confidence level of presence of an ear infection in the ear of the patient; and
outputting an indication including the confidence level for display on a user interface.

2. The method of claim 1, further comprising segmenting the image, and
wherein the input to the machine learning trained model includes each segmented portion of the image.

3. The method of claim 2, further comprising performing object detection on the image to identify a Malleus Handle in the image, and wherein segmenting the image includes using the identified Malleus Handle as an axis for segmentation.

4. The method of claim 1, further comprising performing object detection on the image to identify whether the image captures an entirety of an ear drum of the ear.

5. The method of claim 1, wherein determining the overall confidence level includes multiplying a confidence level output from the machine learning trained model by a confidence level output from the trained classifier.

6. The method of claim 1, wherein receiving the text c des receiving a selection from a list of symptoms.

7. The method of claim 1, wherein receiving the text includes receiving user input custom text.

8. The method of claim 1, wherein the classifier is a support vector machine (SVM) classifier or a logistic regression model classifier.

9. The method of claim 1, wherein the machine learning trained model is a convolutional neural network model.

10. A system for generating an ear disease state prediction to assist diagnosis of an ear disease, the system comprising:
processing circuitry; and
memory including instructions, which when executed, cause the processing circuitry to:
receive, at a processor, an image captured by an otoscope of an inner portion of an ear of a patient;
predict, at the processor, an image-based confidence level of a disease state in the ear by using the image as in input to a machine learning trained model;
receive text corresponding to a symptom of the patient;
predict a symptom-based confidence level of the disease state in the ear by using the text as in input to a trained classifier;
use the results of the image-based confidence level and the symptom-based confidence level to determine an overall confidence level of presence of an ear infection in the ear of the patient; and
output an indication including the confidence level for display on a user interface.

11. The system of claim 10, wherein the instructions further cause the processing circuitry to segment the image, and wherein the input to the machine learning trained model includes each segmented portion of the image.

12. The system of claim 11, wherein the instructions further cause the processing circuitry to perform object detection on the image to identify a Malleus Handle in the image, and wherein segmenting the image includes using the identified Malleus Handle as an axis for segmentation.

13. The system of claim 10, wherein the instructions further cause the processing circuitry to perform object detection on the image to identify whether the image captures an entirety of an ear drum of the ear.

14. The system of claim 10, wherein to determine the overall confidence level, the instructions further cause the processing circuitry to multiply a confidence level output from the machine learning trained model by a confidence level output from the trained classifier.

15. The system of claim 10, wherein to receive the text, the instructions further cause the processing circuitry to receive a selection from a list of symptoms.

16. The system of claim 10, wherein to receive the text, the instructions further cause the processing circuitry to receive user input custom text.

17. The system of claim 10, wherein the classifier is a support vector machine (SVM) classifier or a logistic regression model classifier.

18. The system of claim 10, wherein the machine learning trained model is a convolutional neural network model.

19. At least one machine-readable medium including instructions for generating an ear disease state prediction to assist diagnosis of an ear disease, which when executed by processing circuitry, cause the processing circuitry to perform operations to:
receive, at a processor, an image captured by an otoscope of an inner portion of an ear of a patient;
predict, at the processor, an image-based confidence level of a disease state in the ear by using the image as in input to a machine learning trained model;
receive text corresponding to a symptom of the patient;
predict a symptom-based confidence level of the disease state in the ear by using the text as in input to a trained classifier;
determine, using the results of the image-based confidence level and the symptom-based confidence level, an overall confidence level of presence of an ear infection in the ear of the patient; and
output an indication including the confidence level for display on a user interface.

20. The at least one machine-readable medium of claim 19, wherein the classifier is a support vector machine (SVM) classifier or a logistic regression model classifier and wherein the machine learning trained model is a convolutional neural network model.
